(19) United States
(12) Patent Application Publication
(10) Pub. No.: US 2026/0248811 A1
(43) Pub. Date: Aug. 27, 2026

(54) COMPOSITIONS FOR TREATING AN INDIVIDUAL FOR IMPROVEMENT OF COGNITIVE FUNCTIONS

(71) Applicant: Kim VAN ROOIJ, Amstelveen (NL)

(72) Inventor: Kim VAN ROOIJ, Amstelveen (NL)

(21) Appl. No.: 19/489,657

(22) PCT Filed: Jun. 6, 2024
(86) PCT No.: PCT/NL2024/050295

§ 371 Date: Dec. 3, 2025

(30) Foreign Application Priority Data
Jun. 6, 2023 (NL) 2035008

(51) Int. Cl. A61K 31/568; A61K 38/22; A61N 2/00; A61N 2/02; A61P 25/28

(52) U.S. Cl. CPC A61K 31/568; A61K 38/22; A61N 2/002; A61N 2/006; A61N 2/02; A61P 25/28

(57) ABSTRACT

The invention relates to the field of drug formulation and drug delivery. More specifically, the invention relates to a combination of active ingredients for administration to individuals for improvement of cognitive dysfunction/disorder that can be applied with rTMS.

### FIELD

The invention relates to the field of drug formulation and drug delivery. More specifically, the invention relates to a combination of active ingredients for administration to individuals for improvement of cognitive disorders that can be applied with Repetitive Transcranial Magnetic Stimulation (rTMS) and/or cognitive training.

### INTRODUCTION

In cognitive science and neuropsychology, executive functions (collectively referred to as executive function and/or cognitive control) are a set of cognitive processes necessary for the cognitive control of behavior: selecting and successfully monitoring behaviors that facilitate the attainment of chosen goals. Executive functions include basic cognitive processes such as attentional control, cognitive inhibition, inhibitory control, working memory, and cognitive flexibility. Higher-order executive functions require the simultaneous use of several basic executive functions and include planning and fluid intelligence (for example, reasoning and problem solving).

Repeated transcranial magnetic stimulation (rTMS) is a method in which brain areas are stimulated or inhibited by generating a strong magnetic field in an electromagnet placed on the skull. Functional magnetic resonance imaging (fMRI) studies have shown that the effect of rTMS can induce changes in remote brain regions. In the stimulated regions, low-frequency (≤1 Hz) rTMS can induce inhibitory effects, while high-frequency (≥5 Hz) stimulation can induce excitatory effects. As an example, subthreshold 5-Hz rTMS increases motor cortex excitability in healthy humans. However, the number of pulses may exhibit a ceiling effect in that beyond a certain point, that is, increasing the number of pulses may exhibit no further increase in cortical excitability.

Experimental research in healthy volunteers and patients shows that rTMS can have positive effects on cognitive functions (and improvement of cognitive dysfunctions), such as short and long-term memory and the (aforementioned) executive functions, including working memory.

The current invention now provides for further means and methods that are useful for rTMS and that provide for further and/or advantageous means and methods for use in rTMS.

### SUMMARY OF THE INVENTION

The current invention now sought to advantageously combine rTMS treatments with administration of testosterone, and/or functional analogues thereof, and/or oxytocin, and/or functional analogues thereof. Without being bound by theory, in the present invention because of the different underlying mechanisms of testosterone and/or oxytocin, which may include functional analogues thereof, executive cognitive functions, including working memory, can be highly improved. Such a combinatorial treatment provides for a strong improvement of cognitive functions, which may even be further improved by combining with cognitive training (i.e. a social-cognitive activation program).

Hence, in a first embodiment, testosterone and/or a functional analogue thereof is provided for use in a treatment of a subject suffering from a cognitive condition, the treatment comprising administering an effective dosage of testosterone and/or functional analogue thereof to thereby treat said subject suffering from a cognitive condition. In a further embodiment, testosterone and/or a functional analogue thereof is provided for use in a treatment of a subject suffering from a cognitive condition wherein the treatment further comprises applying repetitive transcranial magnetic stimulation (rTMS) pulses to the brain of the subject, having a stimuli frequency, number of pulses, and intensity sufficient to induce an electric field in the brain of the subject, whereby the rTMS pulses are applied within a period of 3-6 hours after administering the testosterone and/or the functional analogue thereof. In yet another further embodiment, testosterone and/or a functional analogue thereof for use in accordance with the invention is provided wherein the treatment further comprises administering an effective dosage of oxytocin and/or a functional analogue thereof. Instead of referring to a subject suffering from a cognitive condition, one may refer to a subject suffering from a cognitive dysfunction and/or disorder.

Hence, testosterone and/or a functional analogue thereof is provided for use in a treatment of a subject suffering from a cognitive dysfunction/disorder, the treatment comprising administering an effective dosage of testosterone and/or functional analogue thereof to thereby treat said subject suffering from a cognitive dysfunction/disorder. In a further embodiment, testosterone and/or a functional analogue thereof is provided for use in a treatment of a subject suffering from a cognitive dysfunction/disorder wherein the treatment further comprises applying repetitive transcranial magnetic stimulation (rTMS) pulses to the brain of the subject, having a stimuli frequency, number of pulses, and intensity sufficient to induce an electric field in the brain of the subject, whereby the rTMS pulses are applied within a period of 3-6 hours after administering the testosterone and/or the functional analogue thereof. In yet another further embodiment, testosterone and/or a functional analogue thereof for use in accordance with the invention is provided wherein the treatment further comprises administering an effective dosage of oxytocin and/or a functional analogue thereof.

Further embodiments include repeated application of the sequence of steps, and/or immediately followed by subsequent cognitive training. Further embodiments include repeated application of the sequence of steps, and/or followed or preceded by cognitive training. For example, in one embodiment, testosterone and/or a functional analogue thereof is provided for use in accordance invention as defined herein, wherein the treatment comprises
administering the subject with an effective dosage of testosterone and/or a functional analogue thereof,
optionally, and preferably, applying rTMS pulses to the brain of the subject, and/or
optionally, and preferably, administering an effective dosage of oxytocin or a functional analogue thereof,
   wherein the treatment is performed 1 to 7 times a week during a period 1 to 10 weeks, preferably 2-3 times a week for 5 weeks or more.

In yet another further embodiment, provided is testosterone and/or a functional analogue thereof for use in accordance with the invention, wherein the treatment comprises:
administering the subject with an effective dosage of testosterone and/or a functional analogue thereof,
optionally, and preferably, applying rTMS pulses to the brain of the subject, and/or
optionally, and preferably, administering an effective dosage of oxytocin or a functional analogue thereof,
   subsequently further comprises,
exposing the subjects to cognitive training, preferably immediately subsequent to administering of oxytocin.
   In yet another further embodiment, provided is testosterone and/or a functional analogue thereof for use in accordance with the invention, wherein the treatment comprises:
administering the subject with an effective dosage of testosterone and/or a functional analogue thereof,
optionally, and preferably, applying rTMS pulses to the brain of the subject, and/or
optionally, and preferably, administering an effective dosage of oxytocin or a functional analogue thereof,
   subsequently further comprises,
exposing the subjects to cognitive training, preferably during the period that the above treatments are given.

### FIGURES

FIG. 1. Plotted in this graph is measured plasma levels of total testosterone (mmol/L, left graph) and plasma levels of free testosterone (pmol/L, right graph), prior to administration (left bar, before), after testosterone administration (middle bar, T) and after administration of a combination of testosterone and dihydrotestosterone (right bar, T+DHT). The amount of free testosterone is higher with the combination of testosterone and dihydrotestosterone as compared with testosterone alone.

FIG. 2. Plotted is scored cognitive performance from left to right, i.e. overview of activities today, remembering things today, forgotten to do things today, and intended plans implemented today, comparing three different conditions (no drugs (left), chess (middle), and chess+T&O (testosterone and oxytocyin treatment). The no drugs/Chess condition does not involve administration of testosterone and oxytocin nor chess. An upward trend in scoring is observed for overview of activities today, remembering things today, and intended plans implemented today, going from no drugs/no chess, to chess, and to chess &T&O treatment. The forgotten to do things today score showed a downward trend going from no drugs, chess to chess+T&O treatment.

### DEFINITIONS

The term “sublingual formulation”, as is used herein, refers to a formulation of an active ingredient that is suitable for administration under the tongue, also termed hypoglossal or subglossal. A sublingual formulation allows the release of an active ingredient, in the present case a testosterone and/or a functional analogue thereof, when held in the oral cavity such as under the tongue, preferably within minutes, and allows the active ingredient to diffuse through the mucous membrane into the blood. Administration of an active ingredient by a sublingual formulation therefore bypasses the gastro-intestinal tract.

The term “sublingual”, also termed “buccal”, as is used herein, refers to the interspace between the lips and the teeth and the interspace between the cheek and the teeth. The sublingual area is delimited by the palate and tongue and includes the true sublingual area.

The term “cyclodextrin”, as is used herein, includes reference to a displacement enhancer such as hydroxypropylbeta-cyclodextrin, poly-betacyclodextrin or gamma-cyclodextrin.

The terms “treating” and “treatment”, as are used herein, refer to reduction in severity and/or frequency of symptoms, elimination of symptoms and/or underlying cause, retardation of progression of a condition impacting cognition. Thus, for example, “treating” a human involves amelioration of symptoms and treatment of a clinically symptomatic human by inhibiting or causing regression of a condition such as cognitive condition or cognitive impairment.

The term “free testosterone”, as is used herein, refers to a short, sharp peak level of free testosterone of at least 0,010 nmol/l in the blood circulation of the human. A testosterone in the circulation is typically bound by steroid hormone binding globulin (SHBG) and by albumin. It is important that the peak plasma level of testosterone is present and calculated as free testosterone, so a fraction not bound by albumin and SHBG. Hence, the dose of testosterone that is provided should be high enough to saturate the albumin and SHBG (i.e. the concentration of testosterone and/or functional analogue thereof must be high enough to overcome direct, complete binding of testosterone by SHBG or albumin), or another way of avoiding binding to albumin or SHBG must be designed, such as the use of a competitor for the testosterone binding site on SHBG.

### DETAILED DESCRIPTION OF THE INVENTION

In one embodiment, testosterone and/or a functional analogue thereof is provided for use in a treatment of a subject suffering from a cognitive condition, e.g. a cognitive dysfunction/disorder, the treatment comprising administering an effective dosage of testosterone and/or functional analogue thereof to thereby treat said subject suffering from a cognitive condition.

In another embodiment, testosterone and/or a functional analogue thereof is provided for use in a treatment of a subject suffering from a cognitive dysfunction/disorder, the treatment comprising administering an effective dosage of testosterone and/or functional analogue thereof to thereby treat said subject suffering from a cognitive dysfunction/disorder, and further comprising applying rTMS pulses to the brain of the subject, having a stimuli frequency, number of pulses, and intensity sufficient to induce an electric field in the brain of the subject, whereby the rTMS pulses are applied within a period of 3-6 hours after administering the testosterone and/or the functional analogue thereof.

In yet another embodiment, testosterone and/or a functional analogue thereof is provided for use in a treatment of a subject suffering from a cognitive dysfunction/disorder, the treatment comprising administering an effective dosage of testosterone and/or functional analogue thereof to thereby treat said subject suffering from a cognitive dysfunction/disorder, wherein the treatment further comprises administering an effective dosage of oxytocin and/or a functional analogue thereof.

In still yet another further embodiment, testosterone and/or a functional analogue thereof is provided for use in a treatment of a subject suffering from a cognitive dysfunction/disorder, the treatment comprising administering an effective dosage of testosterone and/or functional analogue thereof to thereby treat said subject suffering from a cognitive dysfunction/disorder, wherein the treatment further comprises administering an effective dosage of oxytocin and/or a functional analogue thereof, and further comprising applying rTMS pulses to the brain of the subject, having a stimuli frequency, number of pulses, and intensity sufficient to induce an electric field in the brain of the subject, whereby the rTMS pulses are applied within a period of 3-6 hours after administering the testosterone and/or the functional analogue thereof.

Testosterone is also known under the chemical name 17-β-hydroxyandrost-4-en-3-one. Testosterone can be obtained in various ways: it may be isolated and purified from nature or synthetically produced by any manner. The term “functional analogue of testosterone” as used herein relates to any useful metabolite or precursor of testosterone, for example the metabolite dihydrotestosterone that can provide the same function as testosterone. It is clear to the skilled person that if a metabolite or precursor of testosterone is used, i.e. a functional analogue of testosterone, the time point for administration may be appropriately adjusted if needed, relative to a selected time point of administration of testosterone.

Oxytocin is a nonapeptide and a mammalian hormone. It is normally degraded in the gastrointestinal tract, therefore it is most commonly administered as liquid formulation by injection or as nasal spray. Synthetic oxytocin is commercially available as ready-to-use liquid formulations. “Functional analogues” of oxytocin are also known in the art, such as desamino-oxytocin (Hope et al., J Biol Chem 237 (5): 1563-1566, 1962) and carbetocin (Sweeney et al, Curr Ther Res 47:528-540, 1990), which are also commercially available. Hence, it is understood that a functional analogue of oxytocin includes any useful derivative or precursor of oxytocin that can provide for essentially the same function as oxytocin.

It is understood that the use of testosterone and/or a functional analogue thereof, and, oxytocin and/or a functional analogue thereof, may be preferably for use in treatments of subjects suffering from cognitive impairment or in treatment of subjects identified as being at risk therefor. As cognitive functions may be negatively affected by repeated stressful events, and the treatments in accordance with the invention can include multiple administrations, it is highly preferred that the administration route selected is not invasive. A compound such as testosterone and/or a functional analogue thereof, and oxytocin and/or a functional analogue thereof, is thus preferably provided by a route of administration that is not invasive. For example, motivation for cognitive training should normally not be negatively influenced by invasive routes of administration.

A steroid such as testosterone and/or a functional analogue thereof, is preferably provided by a route of administration, such as oral, buccal or intranasal administration, or by inhalation, for example by employment of a nebulizer. A preferred route of administration of a testosterone and/or a functional analogue thereof is oral administration, for example by a administering liquid formulation or by a sublingual formulation. Said sublingual formulation preferably comprises a displacement enhancer such as, for example, a cyclodextrin. Oral administration of testosterone is a comfortable option with minimal side effects such as accidental transfer of testosterone to women or children that can occur from transdermal products. In addition, oral administration of testosterone may increase adherence to treatment. Hence, in one embodiment, testosterone and/or a functional analogue thereof for use in accordance with the invention is administered in the form of a sublingual formulation, preferably comprising cyclodextrin.

Said testosterone and/or a functional analogue thereof is preferably provided such that a short, sharp peak level of testosterone is present about 10-30 minutes, after administration of the testosterone and/or a functional analogue thereof. Said short, sharp peak level of testosterone preferably is provided as a short, sharp peak level of free testosterone in the blood circulation of the human. Methods for determining the amount of free testosterone are known in the art such as, for example, described in Lavoie et al., 1989 (Lavoie et al., 1989. Clinical Biochemistry 22:451-456). It is understood that the uses and methods as provided herein highly preferably include the treatment of human subjects.

Without being bound by theory, administration of testosterone in accordance with the invention above a certain threshold value causes a pharmacological peak in testosterone. Above that threshold, administration of testosterone is accompanied by an increase in free testosterone. Free testosterone can have a biological effect, and thus behavioral or cognitive effects. An increase in testosterone does not have an immediate effect on certain stimulus-response relationships. An increase in free testosterone in the circulation is associated approximately 3-6 hours later with a greater sensitivity of brain areas to certain stimuli-response effects (it is about stimuli that are sensitive to testosterone, which may include all kinds of cognitive functions). Testosterone administered e.g. sublingually with cyclodextrins (for women about 0.1-2 mg; for men about 0.5-20 mg) causes a pharmacological peak in testosterone and free testosterone after about 15 minutes. Hence, such a pharmacological peak e.g. induced by sublingual testosterone, can cause, e.g. in a period of 3 to 6 hours after administration, a greater sensitivity of the brain to stimuli and therefore an increasing effect on cognitive function.

In accordance with the invention, testosterone and/or a functional analogue thereof may preferably comprise testosterone and/or dihydrotestosterone. It is contemplated in accordance with the invention to use a mixture of testosterone and dihydrotestosterone. A mixture comprising testosterone and dihydrotestosterone preferably comprises said testosterone and dihydrotestosterone in a ratio of between 10:90 and 90:10 (w/w). Said ratio preferably is between 20:80 and 80:20 (w/w), more preferably between 30:70 and 70:30 (w/w), such as between 40:60 and 60:40 (w/w), including about 50:50 and 90:10 (w/w). A more preferred mixture may comprise testosterone and dihydrotestosterone in a ratio of between 10:90 and 90:10 (w/w). Said ratio preferably is between 25:75, about 30:70, such as about 40:60 and 45:55 (w/w).

In one embodiment, the total amount of testosterone and/or a functional analogue thereof, such as dihydrotestosterone, that is provided to an individual in need thereof is between 0.1 and 20 milligram. When the individual is a male, said testosterone and/or a functional analogue thereof, such as dihydrotestosterone, is preferably administered in a total amount of between 0.5 and 20 milligram, preferably between 1 and 10 milligram, including between 5 and 10 milligram. A preferable amount administered to a male can be about 5 mg testosterone. When the individual is a female, said testosterone and/or a functional analogue thereof, such as dihydrotestosterone, is preferably administered in a total amount of between 0.1 and 2 milligram, preferably between 0.25 and 1 milligram, such as between 0.3 and 0.7 milligram, including about 0.4 milligram, about 0.5 milligram and about 0.6 milligram. A preferable amount administered to a female can be about 0.5 mg testosterone.

In one embodiment, the total amount of testosterone and dihydrotestosterone, that is provided to an individual suffering from a cognitive disorder and/or dysfunction is between 0.1 and 20 milligram. When the individual is a male, said testosterone and dihydrotestosterone, is preferably administered in a total amount of between 0.5 and 20 milligram, preferably between 1 and 10 milligram, including between 5 and 10 milligram. When the individual is a female, said testosterone and dihydrotestosterone, is preferably administered in a total amount of between 0.1 and 2 milligram, preferably between 0.25 and 1 milligram, such as between 0.3 and 0.7 milligram, including about 0.4 milligram, about 0.5 milligram and about 0.6 milligram. As shown in the examples herein, a suitable dosage of testosterone and dihydrotestosterone may be about 5 mg testosterone and about 5 mg dihydrotestosterone in male individuals, and about 0.5 mg testosterone and about 0.5 mg dihydrotestosterone in female individuals.

A preferred route of administration of testosterone and/or a functional analogue thereof, such as dihydrotestosterone, is oral administration, preferably by sublingual administration.

Said sublingual formulation is preferably provided as an immediate release delivery system for oral administration that is formulated to provide a peak level of free testosterone of at least 0,020 nmol/l in the blood circulation.

Accordingly, in another embodiment, testosterone and/or a functional analogue thereof, for use in accordance with the invention, is formulated to provide upon administration a peak level of free testosterone of 0,020 nmol/L or more for females, and 0.4 nmol/L or more for males in the blood circulation of the subject. In yet another embodiment, testosterone and/or a functional analogue thereof, for use in accordance with the invention, is formulated to provide upon administration a peak level of free testosterone of 0,020 nmol/L for females, and 0.4 nmol/L for males in the blood circulation of the subject.

A preferred immediate release delivery system for oral administration comprises a core and a coating surrounding the core, said coating comprising said testosterone and and/or a functional analogue thereof, such as dihydrotestosterone, and further comprising a cyclodextrin. Said coating further preferably comprises a cellulose such as agglomerated cellulose, microcrystalline cellulose or a combination thereof. Said cellulose preferably is selected from methylcellulose, hydroxypropyl methylcellulose, and carboxy methyl cellulose, preferably is or comprises hydroxypropyl methylcellulose. The presence of a poorly soluble steroid such as testosterone and/or a functional analogue thereof, such as dihydrotestosterone, and a carrier such as a cyclodextrin provides rapid and efficient delivery of the testosterone and/or a functional analogue thereof, such as dihydrotestosterone, to the mucous membrane, from which the steroid is than rapidly absorbed into the circulation.

The neuropeptide oxytocin acts as a hormone and as a neurotransmitter and is known for its peripheral endocrine effects on labor induction and breast milk production. Oxytocin is also released in the brain, where it modulates various aspects of social behavior. For example, oxytocin promotes maternal nurturing and attachment, enhances social reward, and increases salience of certain social stimuli. Oxytocin improves social memory and bonding behavior in monogamous species. The physiological properties of oxytocin in the human brain are associated with fluctuations in the amount of oxytocin present during different circumstances, diffuse projections of oxytocin in the brain, and there are interactions with other systems such as the dopaminergic and serotonergic systems. These properties indicate that oxytocin lacks clear spatial and temporal specificity, but has widespread effects on intrinsic brain functioning. Studies using functional magnetic resonance imaging (fMRI) with oxytocin administration reveal a specific set of social brain regions through which oxytocin influences human behavior. Oxytocin not only has the potential to modulate activity in a range of specific brain regions, but also the functional connectivity between these regions involved in social behavior (such as pair bonding, sexual behavior, social bonding, etc.). In addition, there is increasing evidence that oxytocin is involved in neurogenesis and neuroplasticity of the brain, which might improve cognitive (executive) functions such as short- and long-term memory. Intranasal administration of oxytocin causes an increase in oxytocin levels in the blood circulation within a few minutes, with the T-max occurring after approximately 15 minutes and returns to baseline after approximately 90 minutes. Means and methods to determine oxytocin levels in the blood are known in the art. Most studies that have investigated oxytocin having effect on behavior and the like use dosages between 20-50 IE of oxytocin, most of them use 24 IE. Behavioral and neural effects of administered dosages are around 15-90 minutes after administration.

There is a time lag for the effect on the brain's sensitivity to cues associated with behavior of the testosterone and/or functional analogue thereof, of about 2.5-6 hours, about 3-6 hours, which may be more specifically around 3-4.5 hours, which may be more specifically around 3.5 to 5 hours, in particular around 4 hours after (sublingual) administration. Furthermore, there is time lag for the effect on the brain's sensitivity to cues associated with behavior of the oxytocin and/or a functional analogue thereof, which is around 15-90 minutes after (intranasal) administration. Hence, testosterone and oxytocin, and/or functional analogues thereof, preferably is not administered at the same time and sublingual administration of testosterone and/or a functional analogue thereof, is preferably performed first, and, subsequently, secondly, oxytocin is administered, intranasally, in such a dosing regimen such that the sensitivity windows for testosterone (e.g. from about 2.5-6 or 3-6 hours after sublingual administration) and oxytocin (e.g. from about 15 minutes to 90 minutes after intranasal administration) overlap.

Hence, in one embodiment, administration of sublingual testosterone and/or a functional analogue thereof is followed by administering of intranasal oxytocin and/or a functional analogue thereof during a time window of about 3-6 hours after the peak concentration of testosterone in the blood circulation. Intranasal oxytocin may be preferably administered about 3.5 hours after the peak of testosterone, this way, the increased value of oxytocin associated with cognitive or other effects coincides with the period of highest behavioral effect of testosterone. In one embodiment, it is preferred to apply rTMS treatment 15-90 minutes after (intranasal) administration of oxytocin.

Hence, in another embodiment, any which way of administration selected for both testosterone and oxytocin, the administration of testosterone and/or a functional analogue thereof is to first provide a peak concentration of testosterone in the blood circulation, and second, the administration of oxytocin and/or a functional analogue thereof, is to provide a peak concentration of oxytocin and/or a functional analogue thereof, said peak concentrations separated from each other by about 3-4 hours. It is understood that rTMS may be initiated, immediately, or shortly (e.g. within 15 minutes, or less) after the peak concentration of oxytocin and/or a functional analogue thereof is achieved. Preferably, this is selected to be within 15 min-90 min after intranasal oxytocin administration.

As said, oxytocin is currently registered as a nasal spray, e.g. with a dose of 4 IE per spray and in containers carrying 50 doses. This is the preferred route of administration as it is relatively convenient. Nevertheless, further suitable forms of oxytocin and/or further suitable means may be contemplated in accordance with the invention that can provide for an effect as observed with intranasally administered oxytocin and that may be suitable for the highly advantageous uses and methods in accordance with the invention, i.e. provide for likewise behavioral and neural effects as observed with intranasal administration. One may even contemplate intravenous administration. Highly advantageously, it may also be contemplated to administer oxytocin in a limited sustained release form, sublingual form, intramuscular injection, inhalation, or, through a hydrogel. Although such alternative means of administration may currently not be registered, such forms have been developed and such forms may provide highly useful alternative means of administration of oxytocin.

In another embodiment, testosterone and/or a functional analogue thereof is provided for use in accordance with the invention, wherein the treatment further comprises administering an effective dosage of oxytocin and/or a functional analogue thereof, wherein said effective dosage of oxytocin and/or a functional analogue thereof is administered in a form providing increased oxytocin levels 3-6 hours after administration of the testosterone and/or the functional analogue.

In another embodiment, testosterone and/or a functional analogue thereof is provided for use in accordance with the invention, wherein the treatment further comprises administering an effective dosage of oxytocin and/or a functional analogue thereof, wherein said effective dosage of oxytocin and/or a functional analogue thereof is administered intranasally, which is preferably administered 3-4 hours after administration of the testosterone and/or the functional analogue. More preferably said administration is about 3 hours and 30 minutes after administration of the testosterone and/or the functional analogue thereof, wherein most preferably, said testosterone and/or functional analogue is administered sublingually.

In yet another embodiment, testosterone and/or a functional analogue thereof for use in accordance with the invention, wherein the treatment further comprises administering an effective dosage of oxytocin and/or a functional analogue thereof, wherein said effective dosage of oxytocin and/or a functional analogue thereof is administered in a total amount in the range of 10-100 IE, preferably in the range of 20-50 IE. It is understood that such amounts preferably relate to intranasally administered amounts of oxytocin and/or a functional analogue, and alternative administration routes may involve equivalent amounts. A suitable amount for administration that may be selected is 24 IU. Suitable amounts for administration in accordance with the invention that may be selected include about 24 IE of ocytocin, and about 5 mg of testosterone for a male subject, and about 0.5 mg of testosterone for a female subject. Suitable amounts for administration in accordance with the invention that may be selected include 24 IE of ocytocin, and 5 mg of testosterone for a male subject, and 0.5 mg of testosterone for a female subject, such as shown in the example section herein, as described herein.

In yet another embodiment, testosterone and/or a functional analogue thereof is provided for use in a treatment in accordance with the invention, wherein the treatment comprises administering an effective dosage of oxytocin and/or a functional analogue thereof, and wherein the treatment further comprises applying repetitive transcranial magnetic stimulation (rTMS) pulses to the brain of the subject, having a stimuli frequency, number of pulses, and intensity sufficient to induce an electric field in the brain of the subject, wherein the rTMS pulses are applied within a period of 3 hours-6 hours, more 3 hours and 45 minutes-5 hours and 15 minutes, more preferably 4-5 hours after the administration of testosterone and/or functional analogue thereof, wherein more preferably, the administration of oxytocin and/or functional analogue thereof is administered about 3 and a half hours after the administration testosterone and/or the functional analogue thereof. Most preferably, sublingual testosterone is administered, and subsequently intranasal oxytocin is administered about 3.5 hours thereafter, wherein subsequently rTMS is applied in the time window of about 3 hours and 45 minutes to 5 hours and 15 minutes after initial sublingual administration, the latter more preferably carried out in the time window of between 4 hours and 5 hours.

As outlined herein, with regard to applying rTMS, different means and methods can be used to advantageously apply pulses of rTMS, i.e. including targeting different brain regions, different frequencies, different numbers and different intensities.

Hence, in a further embodiment in accordance with the invention, testosterone and/or a functional analogue thereof is provided for use as described herein, wherein the rTMS pulses are applied left, right or bilaterally applied to the prefrontal cortex. In a further embodiment in accordance with the invention, testosterone and/or a functional analogue thereof is provided for use as described herein, wherein the rTMS pulses are applied left, right or bilaterally applied to the prefrontal cortex, including the dorsolateral prefrontal cortex (DLPFC) and/or inferior frontal gyrus (IFG). In yet a further embodiment in accordance with the invention, testosterone and/or a functional analogue thereof is provided for use as described herein, wherein the rTMS pulses are applied left, right or bilaterally applied to the dorsolateral prefrontal cortex (DLPFC) and/or inferior frontal gyrus (IFG).

In another embodiment, the rTMS pulses may be selected to have a stimuli frequency of 1-100 HZ. In another embodiment, the rTMS pulses applied include high-frequent rTMS (hz>5) over the left DLPFC and/or low-frequent rTMS (hz<1) at the right DLPFC, and/or high frequent rTMS targeting the right IFG. In yet another embodiment, the rTMS pulses applied include high-frequent rTMS (hz>5) over bilateral DLPFC. In still another embodiment, the number of rTMS pulses is selected from the range of 100-3000. In yet another embodiment, the stimulus intensity of the applied rTMS is selected from the range between 0.05 and 5 Tesla.

As the means and methods in accordance with the invention relate to cognitive functions, it is understood that patients suffering from such dysfunction/disorders (or subjects suspected to suffer therefrom) may benefit from multiple treatments.

Hence, in a further embodiment, testosterone and/or a functional analogue thereof is provided for use in accordance with the invention as described herein, comprising administering an effective dosage of testosterone and/or a functional analogue thereof, as described herein,
and subsequently applying rTMS pulses to the brain of the subject, as described herein, which are performed 1 to 7 times a week during a period 1 to 10 weeks, preferably 2-3 times a week for 5 weeks or more.

Hence, in a further embodiment, testosterone and/or a functional analogue thereof is provided for use in accordance with the invention as described herein, comprising administering an effective dosage of testosterone and/or a functional analogue thereof, as described herein,
subsequently administering an effective dosage of oxytocin and/or a functional analogue thereof, as described herein,
and, subsequently applying rTMS pulses to the brain of the subject, as described herein, wherein these steps are is performed 1 to 7 times a week during a period 1 to 10 weeks, preferably 2-3 times a week for 5 weeks or more.

It is understood that after applying rTMS, subsequently thereafter, preferably immediately thereafter, subjects are subjected to cognitive training.

Hence, in another and/or a further embodiment the treatments as defined herein comprise:
administering the subject with an effective dosage of testosterone and/or a functional analogue thereof, as defined herein,
optionally, and preferably, administering an effective dosage of oxytocin or a functional analogue thereof,
optionally, and preferably, applying rTMS pulses to the brain of the subject,
and subsequently further comprises,
   exposing the subjects to cognitive training during the period that the above treatments are given. As said, preferably said exposure to cognitive training is an immediate subsequent exposure or during the period that the above treatments are given. Suitable cognitive training may include training cognitive and/or executive functions, such as by learning or practicing chess. Accordingly, the treatments, including the treatment schedules with regular treatments and cognitive training highly advantageously allow improving cognitive function. Improving cognitive function in accordance with the invention may include improving working memory.

Improvements in working memory can be measured with a variety of different tests. As a non-limiting example, the n-back test may be applied to test and confirm improvements in working memory which was introduced by Wayne Kirchner in 1958. The n-back task is a continuous performance task that is commonly used as an assessment in psychology and cognitive neuroscience to measure a part of working memory and working memory capacity. The subject is presented with a sequence of stimuli, and the task consists of indicating when the current stimulus matches the one from n steps earlier in the sequence. The load factor n can be adjusted to make the task more or less difficult. In any case, any suitable test may be selected to confirm improvement in working memory. In addition, the treatment in accordance with the invention may also be highly useful for the treatment of a subject suffering from a cognitive dysfunction/disorder, wherein the treatment comprises halting or delaying progression of memory impairment.

As is understood from the disclosure, not only is testosterone and/or a functional analogue thereof, provided in accordance with the invention for use in the treatment of a cognitive dysfunction/disorder as described herein. The invention furthermore provides for oxytocin and/or a functional analogue thereof, for use in a treatment of a subject suffering from a cognitive dysfunction/disorder, as described herein.

Furthermore, the invention provides for oxytocin and/or a functional analogue thereof, for use in a treatment of a subject suffering from a cognitive dysfunction/disorder, as defined herein, wherein said treatment comprises administering an effective dosage of testosterone and/or a functional analogue thereof, and, administering an effective dosage of oxytocin and a functional analogue thereof, to thereby treat said subject suffering from a cognitive dysfunction/disorder. It is understood that such treatments highly preferably include the application of rTMS as defined herein.

Furthermore, the invention provides for oxytocin and/or a functional analogue thereof, for use in a treatment of a subject suffering from a cognitive dysfunction/disorder, as defined herein, wherein said treatment comprises administering an effective dosage of testosterone and/or a functional analogue thereof, as defined herein, and, administering an effective dosage of oxytocin and a functional analogue thereof, as defined herein. to thereby treat said subject suffering from a cognitive dysfunction/disorder. It is understood that such treatments highly preferably include the application of rTMS as defined herein.

Furthermore, the invention provides for testosterone and/or a functional analogue thereof, and, oxytocin and/or a functional analogue thereof, for use in a treatment of a subject suffering from a cognitive dysfunction/disorder, wherein said treatment comprises administering an effective dosage of testosterone and/or a functional analogue thereof, and, administering an effective dosage of oxytocin and a functional analogue thereof, to thereby treat said subject suffering from a cognitive dysfunction/disorder. It is understood that this treatment highly preferably includes the application of rTMS as defined herein.

Furthermore, the invention provides for testosterone and/or a functional analogue thereof, and, oxytocin and/or a functional analogue thereof, for use in a treatment of a subject suffering from a cognitive dysfunction/disorder, wherein said treatment comprises administering an effective dosage of testosterone and/or a functional analogue thereof, as defined herein, and, administering an effective dosage of oxytocin and a functional analogue thereof, as defined herein, to thereby treat said subject suffering from a cognitive dysfunction/disorder. It is understood that this treatment highly preferably includes the application of rTMS as defined herein.

Cognitive performance, in subjects suffering from a cognitive condition, i.e. a cognitive dysfunction/disorder, can be improved by subjecting subjects to the treatments as contemplated herein, such as a treatment comprising a combination of testosterone and oxytocin, or the like, further combined with cognitive training and/or rTMS. Cognitive performance is improved, when subjecting subjects to the treatments as contemplated herein, with regard to having an improved overview of activities, improved memory (remembering more things and forgetting less), or improved planning, as compared with no treatment at all and as compared with only cognitive training. Cognitive performance is improved, when subjecting subjects to the treatments as contemplated herein, with regard to one or more of having an improved overview of activities, improved memory (remembering more things and forgetting less), and improved planning, as compared with no treatment at all and as compared with only cognitive training.

### EXAMPLES

Testosterone (T) vs. testosterone and dihydrotestosterone (T+DHT) The first example concerns two male heterosexual patients (64 and 67 years). Two alternative forms of testosterone administration were considered for a treatment. The first treatment involved taking 5 mg sublingual testosterone (T) (solution consisting of alcohol, denaturalized water, testosterone and cyclodextrins). The second consisted of simultaneously taking 5 mg sublingual testosterone and 5 mg sublingual dihydrotestosterone (DHT) (solution consisting of alcohol, denaturalized water, DHT and cyclodextrins). The testosterone and DHT solutions were prepared by a pharmacist on the request of a general practitioner. Subsequently, the changes in total testosterone and free testosterone in blood plasma were examined, 15 minutes after intake of the androgen(s).

The table below describes the effects of the different treatments on ‘total testosterone’ and ‘free testosterone (i.e. measured, not calculated)’ for both patients:

| | | | | Total T | Free T | SHBG | PSA |
|---|---|---|---|---|---|---|---|
| | | DOT | date | nmol/L | pml/L | nmol/L | μmol/L |
| Pa- | No Drug | 1 | 7-3 | 10.7 | 190 | 35 | 0.60 |
| tient | T | 1 | 7-3 | 28.7 | 440 | | |
| 1 | T + DHT | 2 | 10-3 | 28.8 | 560 | | |
| Pa- | No Drug | 1 | 7-3 | 11.1 | 220 | 29 | 1.80 |
| tient | Testosterone | 1 | 7-3 | 43.9 | 1030 | | |
| 2 | (T) | | | | | | |
| | T + DHT | 2 | 10-3 | 44.2 | 1096 | | |

T = testosterone
DHT = dihydrotestosterone

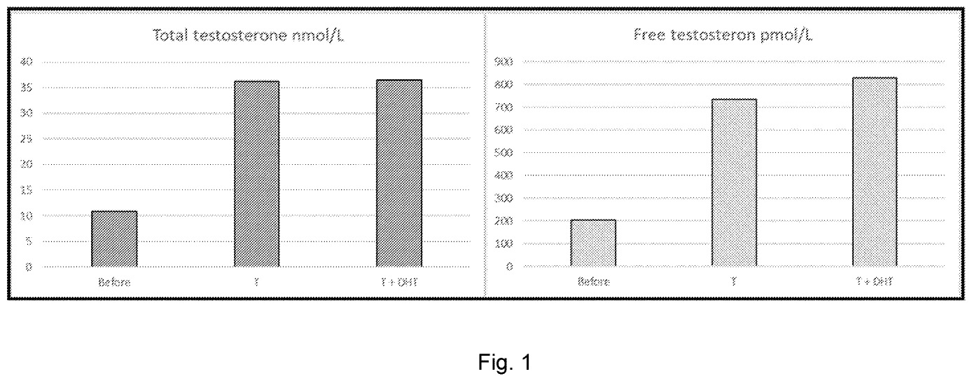

These results are summarized in FIG. 1, depicting the average of the two patients. The results shown in the table and figure show that sublingual T and sublingual T & DHT have an equal effect on total testosterone (left figure), while sublingual T+DHT (compared to T alone) appears to have a greater effect on free testosterone (right figure). This is consistent with the assumption that SHBG becomes more saturated as a result of the binding of DHT to SHBG. This means that the ingested testosterone can no longer bind to SHBG, and consequently there will be a greater increase in unbound testosterone (i.e. free testosterone). By combining sublingual T with DHT, free testosterone in plasma is higher compared to sublingual T alone (as stated above). Consequently, by a combination of T and DHT a lower dosage of sublingual T can be used to reach the same level of free testosterone as compared to sublingual T alone. Total testosterone and free testosterone were measured 15 minutes after the administration of T and (T&DHT). dysfunction/disorder

### Treatment of Cognitive Dysfunctions

Results are shown below of the influence of on the one hand a cognitive activation program (in the current case through chess) and on the other hand a cognitive activation program combined with 5 mg (man) and 0.5 mg (woman) of sublingual testosterone, followed by 3.5 hours later by 24 IU intranasal oxytocin.

Patients were a man and a woman (both middle-aged) (partners) who had both reported complaints of forgetfulness and a loss of overview of daytime activities. They were afraid of the onset of dementia. The following treatment was proposed and carried out. Both patients learned to play chess and played chess for 7 evenings (1 to 1.5 hours) (because chess requires all executive cognitive functions including working memory). They then played chess another evening (8th evening), but this time after administration of sublingual testosterone followed by intranasal oxytocin (3.5 hours after testosterone administration). Patients were asked to start playing chess on this medication day approximately 15 to 30 minutes after oxytocin administration. The patients were asked a number of subjective questions to monitor whether these activities showed improvement in their cognitive complaints. Patients were asked to complete this questionnaire once before the chess program, once the evening after the seventh chess evening (so evening 7 was the last chess evening, the questions were answered the evening after the last chess evening) and once the evening after the chess evening under condition of administration of sublingual testosterone and oxytocin.

The questionnaire below was used:

### Memory and Cognition Questionnaire

1. On a scale of 1 to 10 (not at all to very much), would you indicate how much overview you had of the things you did today? Please circle.
Not at all [1-2-3-4-5-6-7-8-9-10] Very much
2. On a scale of 1 to 10 (not at all well to very well), would you indicate how well you remembered things today? Please circle.
Not at all [1-2-3-4-5-6-7-8-9-10] Very well
3. On a scale of 1 to 10 (not at all to very often), would you indicate whether you forgot anything you wanted to do today? Please circle.
Not at all [1-2-3-4-5-6-7-8-9-10] Very much
4. On a scale of 1 to 10 (from not at all to very much) would you indicate whether you were able to do all the plans you had today? Please circle.
Not at all [1-2-3-4-5-6-7-8-9-10] Very much

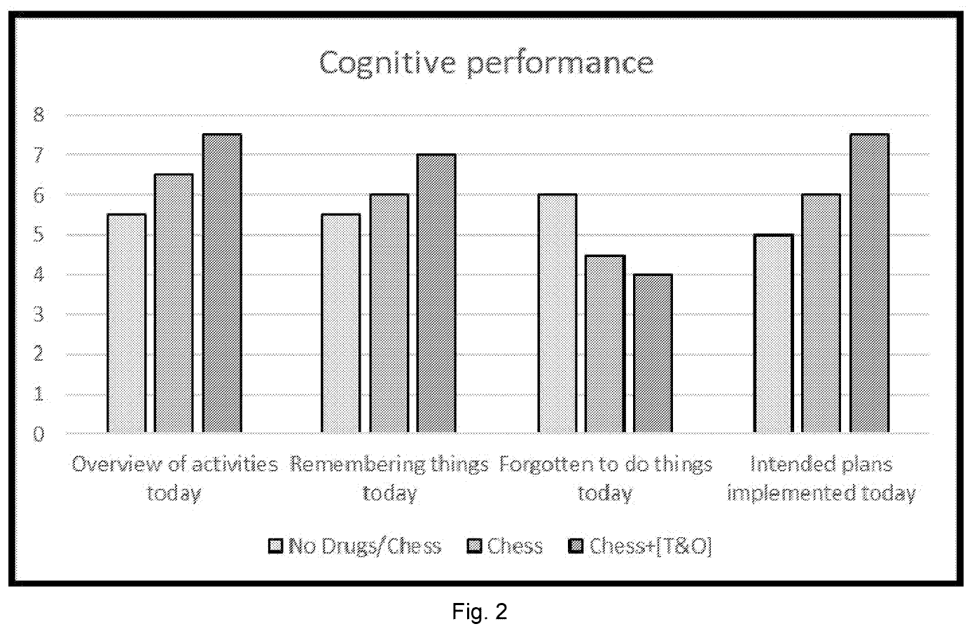

FIG. 2 shows the subjective experience of cognitive functioning under the influence of the different treatments (average of both patients).

The results shown in FIG. 2 indicate that a cognitive activation program combined with a treatment of sublingual testosterone & oxytocin has positive effects on (at least) the subjective experience of cognitive functioning.

Likewise, patients are treated with a cognitive activation program combined with a treatment of sublingual testosterone and dihydrotestosterone and oxytocin.

As shown in the examples above, in further embodiments, treatments as described herein of a subject suffering from a cognitive dysfunction/disorder, improves memory, reduces forgetting things, improves planning, or improves having overview of activities in treated subjects. Such improvements were obtained as compared while not being subjected to the treatment. Treatment preferably comprise cognitive training (like learning and playing chess), administration of sublingual testosterone and/or functional analogue thereof (like dihydrotestosterone), combined with oxytocin, such as described herein.

Based on these results it is hypothesized that the improvement of cognitive function as a result of cognitive training combined with the treatment with sublingual testosterone and oxytocin causes an increase in functional neuronal activity. Such improvement of cognitive function is likewise obtained with cognitive training combined with the treatment with sublingual testosterone, dihydrotestosterone and oxytocin.

By combining sublingual T, oxytocin and cognitive activation programs with the use of repetitive transcranial magnetic stimulation (rTMS), functional neuronal activity can be further increased. Said combination with rTMS improving cognitive functioning in subjects with a cognitive dysfunction/disorder.

1. A method of treatment of a subject suffering from a cognitive dysfunction/disorder, the treatment comprising administering an effective dosage of testosterone and/or functional analogue thereof to thereby treat said subject suffering from a cognitive dysfunction/disorder.

2. The method of claim 1, wherein the treatment further comprises
applying repetitive transcranial magnetic stimulation (rTMS) pulses to the brain of the subject, having a stimuli frequency, number of pulses, and intensity sufficient to induce an electric field in the brain of the subject,
whereby the rTMS pulses are applied within a period of 3-6 hours after administering the testosterone and/or the functional analogue thereof.

3. The method of claim 1, wherein the treatment further comprises
administering an effective dosage of oxytocin and/or a functional analogue thereof.

4. The method of claim 1, wherein testosterone and/or a functional analogue is administered in the form of a sublingual formulation.

5. The method of claim 1, wherein testosterone and/or a functional analogue is administered in a total amount in the range of 0.1-20 mg.

6. The method of claim 1, wherein the subject is a female subject and wherein said testosterone and/or a functional analogue are administered in a total amount in the range of 0.1-2 mg, or, wherein the subject is a male subject and wherein said testosterone and/or a functional analogue are administered in a total amount in the range of 0.5-20 mg.

7. (canceled)

8. The method of claim 1, wherein, whereby the testosterone and/or the functional analogue thereof is formulated to provide upon administration a peak level of free testosterone of 0.020 nmol/L or more for females, and 0.4 nmol/L for males in the blood circulation of the subject.

9. (canceled)

10. The method of claim 1, wherein the functional analogue of testosterone is dihydrotestosterone.

11. The method of claim 3, wherein said effective dosage of oxytocin and/or a functional analogue thereof is administered in a form providing increased oxytocin levels 3-6 hours after administration of the testosterone and/or the functional analogue.

12. The method of claim 3, wherein said effective dosage of oxytocin and/or a functional analogue thereof is administered intranasally.

13. The method of claim 1, wherein the treatment further comprises administering an effective dosage of oxytocin and/or a functional analogue thereof, wherein said effective dosage of oxytocin and/or a functional analogue thereof is administered in a total amount in the range of 10-100 IE.

14. The method of claim 1, wherein the treatment comprises administering an effective dosage of oxytocin and/or a functional analogue thereof, and wherein the treatment further comprises applying repetitive transcranial magnetic stimulation (rTMS) pulses to the brain of the subject, having a stimuli frequency, number of pulses, and intensity sufficient to induce an electric field in the brain of the subject, wherein the rTMS pulses are applied within a period of 4-5 hours after the administration of testosterone and/or functional analogue thereof, and/or within a period of 2 hours after the administration of oxytocin and/or functional analogue thereof.

15. The method of claim 2, wherein the rTMS pulses are applied left, right or bilaterally applied to the prefrontal cortex, including the dorsolateral prefrontal cortex (DLPFC) and/or inferior frontal gyrus (IFG).

16. The method of claim 2, wherein applying repetitive transcranial magnetic stimulation (rTMS) pulses is performed using a method selected from the group consisting of applying a stimuli frequency of 1-100 HZ; applying a high-frequent rTMS (hz>5) over the left DLPFC; applying a low-frequent rTMS (hz<1) at the right DLPFC; applying a high frequent rTMS targeting the right IFG; applying a high-frequent rTMS (hz>5) over bilateral DLPFC, or a combination thereof.

17-18. (canceled)

19. The method of claim 2, wherein the number of rTMS pulses is 100-3000 and/or the stimulus intensity of the applied rTMS pulses is between 0.05 and 5 Tesla.

20. (canceled)

21. The method of claim 1, wherein the treatment comprises:
administering the subject with an effective dosage of testosterone and/or a functional analogue thereof,
applying rTMS) pulses to the brain of the subject, and
administering an effective dosage of oxytocin or a functional analogue thereof, wherein the administering and applying steps are performed 1 to 7 times a week during a period 1 to 10 weeks, or 2-3 times a week for 5 weeks or more, and subsequently exposing the subject to cognitive training.

22. (canceled)

23. The method of claim 21, wherein the subsequent exposure to cognitive training is an immediate subsequent exposure and comprises at least one of a training session including cognitive functions and a training session including executive functions.

24. (canceled)

25. The method of claim 1, wherein said treatment of a subject suffering from a cognitive dysfunction/disorder comprises improving cognitive function and/or improving working memory in the subject.

26. (canceled)

27. The method of claim 1, wherein said treatment of a subject suffering from a cognitive dysfunction/disorder comprises halting or delaying progression of memory impairment.

28-29. (canceled)

30. The method of claim 2, wherein the treatment further comprises administering an effective dosage of oxytocin and/or a functional analogue thereof.
